(19) United States
(12) Patent Application Publication
(10) Pub. No.: US 2023/0305110 A1
(43) Pub. Date: Sep. 28, 2023

(54) SYSTEM AND METHOD FOR DETECTING OBJECT ABNORMALITY SYMPTOM BASED ON RADAR MICRO-DOPPLER

(71) Applicant: ELECTRONICS AND TELECOMMUNICATIONS RESEARCH INSTITUTE, Daejeon (KR)

(72) Inventors: Won Kyu CHOI, Daejeon (KR); Se Han KIM, Daejeon (KR); You Jin KIM, Daejeon (KR); Jae-young JUNG, Daejeon (KR)

(21) Appl. No.: 17/981,621

(22) Filed: Nov. 7, 2022

(30) Foreign Application Priority Data
Mar. 28, 2022 (KR) 10-2022-0037965

(51) Int. Cl. G01S 7/41; G01S 13/00; G01S 13/72

(52) U.S. Cl. CPC G01S 7/415; G01S 13/003; G01S 13/723; A61B 5/05

(57) ABSTRACT

The present disclosure relates to a system and method for detecting an object abnormality symptom based on a radar micro-Doppler. The system for detecting an object abnormality symptom based on a radar micro-Doppler includes a multiple input multiple output (MIMO) system configured to determine whether an object has a suspicious abnormality symptom by collecting information of the object and a beamforming antenna system configured to form an antenna radiation beam toward the object based on a result of the determination for the object having the suspicious abnormality symptom and to obtain three-dimensional (3D) micro-Doppler information.

### CROSS-REFERENCE TO RELATED APPLICATION

This application claims priority to and the benefit of Korean Patent Application No. 10-2022-0037965 filed on Mar. 28, 2022, the disclosure of which is incorporated herein by reference in its entirety.

### BACKGROUND

### 1. Technical Field

The present disclosure relates to a system and method for detecting an object abnormality symptom based on a radar micro-Doppler.

### 2. Description of Related Art

Today, a technology for preventing the spread of a disease by early discovering an object having a suspicious abnormality symptom and predicting a degree of risk of the object throughout the pandemic period grows in importance.

In order to determine an abnormality symptom of an object, a method using an infrared-based thermal imaging camera has been proposed. Such an infrared-based technology has many limitations in its use because the technology basically determines an abnormality symptom based on a temperature and can be operated only in a line-of-sight.

### SUMMARY

Various embodiments are directed to a system and method for detecting an object abnormality symptom, which can predict an abnormality symptom and a degree of risk of an object by collecting frequency information of a micro-Doppler and constructing the collected information in the form of time-series data.

In an embodiment, a system for detecting an object abnormality symptom based on a radar micro-Doppler includes a multiple input multiple output (MIMO) system configured to determine whether an object has a suspicious abnormality symptom by collecting information of the object, and a beamforming antenna system configured to form an antenna radiation beam toward the object based on a result of the determination for the object having the suspicious abnormality symptom and to obtain three-dimensional (3-D) micro-Doppler information.

The MIMO system includes a transmission antenna and a reception antenna, and tracks a moving path of the object by using a channel transfer function in relation to a signal which is transmitted by the transmission antenna and received by the reception antenna.

The beamforming antenna system determines a degree of risk of the object by forming the antenna radiation beam in a situation in which a line-of-sight has been secured, constructing time-series data by using the 3-D micro-Doppler information, and obtaining features of the abnormality symptom of the object which are changed over time.

The beamforming antenna system determines the degree of risk of the object by a deep learning model using the time-series data as input data.

The beamforming antenna system analyzes the changed features of the abnormality symptom of the object in a time axis by performing spectrogram analysis.

The beamforming antenna system includes a combiner configured to compensate for a phase delay based on an interval between antenna elements disposed in an array form, incident angle information of a radio wave, and a propagation constant.

In an embodiment, the system for detecting an object abnormality symptom based on a radar micro-Doppler further includes a controller configured to receive a result of a determination for whether a device properly operates from the beamforming antenna system and to generate an operation control signal for the device based on the result of the determination.

In an embodiment, a method of detecting an object abnormality symptom based on a radar micro-Doppler includes steps of (a) detecting an object that enters a space and tracking a moving path of the object and (b) forming an antenna radiation beam toward the object, collecting and analyzing a three-dimensional (3-D) micro-Doppler signal of the object, and confirming whether the object has an abnormality symptom.

The step (a) includes tracking the moving path by using a channel transfer function matrix of a multiple input multiple output (MIMO) system.

The step (b) includes confirming whether the object has the abnormality symptom in a way to form the antenna radiation beam by using a beamforming antenna system a line-of-sight of which has been secured and to construct time-series data based on the obtained 3-D micro-Doppler signal.

The step (b) includes forming the antenna radiation beam by compensating for phase delays based on an interval between antenna elements, an incident angle of a radio wave, and a wavelength of a center frequency.

In an embodiment, the method of detecting an object abnormality symptom based on a radar micro-Doppler further includes a step (c) of checking whether a device within the space properly operates by using at least any one of sensing information, a radar cross-section, or the micro-Doppler signal and generating a control command signal for the device.

According to the present disclosure, it is possible to block the spread of a disease by early discovering an object (e.g., a person) having a suspicious abnormality symptom in an environment in which multiple objects (e.g., persons) are crowded and predicting a degree of risk of the object (e.g., person) having the suspicious abnormality symptom.

According to the present disclosure, it is possible to prevent the spread of a disease, such as a contagious disease, in a way to maintain a comfortable environment by controlling a device (e.g., a window or a fan) to operate in its optimum state based on environment information.

Effects of the present disclosure which may be obtained in the present disclosure are not limited to the aforementioned effects, and other effects not described above may be evidently understood by those skilled in the art from the following description.

### BRIEF DESCRIPTION OF THE DRAWINGS

FIG. 1 illustrates an abnormality symptom detection service according to an embodiment of the present disclosure.

FIG. 2 illustrates the tracking of an object location and a channel transfer function matrix based on an MIMO system.

FIG. 3 illustrates the steering of beams of a beamforming antenna system.

FIG. 4 illustrates a process of detecting object abnormality based on a 3-D micro-Doppler signal according to an embodiment of the present disclosure.

FIG. 5 illustrates the acquisition of a micro-Doppler signal of the object having a suspicious abnormality symptom and a deep learning model having input data including 3-D time-series data according to an embodiment of the present disclosure.

FIG. 6 illustrates an example of a time-series micro-Doppler signal of a radar.

FIG. 7 illustrates an example in which 3-D micro-Doppler features are converted into time-series data.

FIG. 8 is a block diagram illustrating a computer system for implementing a method according to an embodiment of the present disclosure.

### DETAILED DESCRIPTION

The aforementioned object and other objects, advantages, and characteristics of the present disclosure and a method for achieving the objects, advantages, and characteristics will be clearly described through the following embodiments with reference to the accompanying drawings.

However, the present disclosure is not limited to the following embodiments, but may be implemented in various shapes different from each other, and the following embodiments are only provided to easily deliver the purposes, configurations, and effects of the present disclosure to those skilled in the art to which the present disclosure pertains. Therefore, the scope of the present disclosure is defined by claims.

Terms used in this specification are used to describe embodiments and are not intended to limit the present disclosure. In this specification, an expression of the singular number includes an expression of the plural number unless clearly defined otherwise in the context. The term “comprises” and/or “comprising” used in this specification does not exclude the presence or addition of one or more other elements, steps and/or devices in addition to a mentioned element, step and/or device.

Like today, a situation in which social activities are reduced and offline activities are substituted with online activities throughout the pandemic period continues. If such a situation continues, social classes who are neglected educationally and economically are increased. There is concern that the nation's growth momentum will be threatened in the long run. Social activities need to continue in line with an international atmosphere called With COVID-19, but preparation for the spread of a disease needs to be considered.

In order to prevent the spread of a disease, a technology for early discovering an object having a suspicious abnormality symptom and predicting a degree of risk of the object is important. However, a method using an infrared-based thermal imaging camera has limitations in that the method can be operated only in a line-of-sight.

Accordingly, there is proposed a technology for determining an abnormality symptom by monitoring the momentum of an object (e.g., a person or a thing) through the analysis of a radar Doppler frequency feature. The radar technology is a technology for finding features of an object by using a difference between frequencies of a transmission signal and a signal that is reflected by the object and then received.

The frequency of a signal, which is reflected by an object that periodically exercises and moves and then received, is different from that of a transmission signal. A different frequency between the transmission signal and the reception signal is called a Doppler frequency. A Doppler frequency that is measured by a radar system may be used as a parameter for obtaining motion information including the speed of a corresponding object.

In order to precisely determine a motional state of an object (e.g., a person or a thing), a micro-Doppler signal in addition to a Doppler needs to be synthetically analyzed. A Doppler frequency that is generated due to a fine motion occurring between macroscopic motions of an object is called a micro-Doppler frequency.

A micro-Doppler signal is used to obtain information on a rotary motion of an object (e.g., a person or a thing) or a small motion of the object. The micro-Doppler signal is widely used to recognize an object (e.g., a person or a thing) and to analyze a motion feature or a motion pattern. Various motion features of an object can be detected by analyzing spectrogram for a micro-Doppler signal.

A common radar-based technology for collecting a micro-Doppler signal collects a one-dimensional (1-D) micro-Doppler signal of an object that exercises with respect to one plane by using a single MIMO or beamforming technology.

Furthermore, in order to separate transmission and reception signals of the radar, a transmission antenna and a reception antenna are spatially separated and operated. Accordingly, it is impossible to determine an accurate abnormality symptom because momentum occurring at the rear of the object cannot be detected and to obtain a micro-Doppler signal if a line-of-sight is not formed between the radar and the object.

The present disclosure has been proposed to solve such a problem, and is intended to predict an abnormality symptom and a degree of risk of an object by collecting three-dimensional (3-D) frequency information of a micro-Doppler for an object through a plurality of beamforming antenna systems and constructing the collected information in the form of time-series data. More specifically, after movements/motions of multiple objects are tracked by using a multiple input multiple output (MIMO) technology, whether an object is abnormal is precisely determined by collecting 3-D micro-Doppler data of the object by using the plurality of beamforming antenna systems if the object is suspected to have an abnormality symptom. Furthermore, a degree of risk of the object is predicted by constructing the collected 3-D micro-Doppler signal in the form of time-series data. A degree of risk of an abnormality symptom can be predicted in association with a deep learning model for collecting a micro-Doppler signal over time and receiving, as an input, 3-D time-series data that has a change in the frequency over time as features. Furthermore, moving paths of multiple objects (e.g., persons) can be monitored by networking the MIMO system and the beamforming antenna systems. A 3-D micro-Doppler signal of an object (e.g., a person) having a suspicious abnormality symptom can be extracted.

According to the present disclosure, an abnormality symptom of an object can be detected based on a Doppler frequency that is reflected by the object and received by a radar. In addition to the detection, the abnormality symptom of the object can also be predicted based on a 3-D micro-Doppler signal. Furthermore, there is an advantage in that an action can be taken against the spread of a contagious disease at an early stage by previously detecting and monitoring an object having a suspicious abnormality symptom in preparation for a case in which the contagious disease is spread due to the activities of a carrier of harmful viruses.

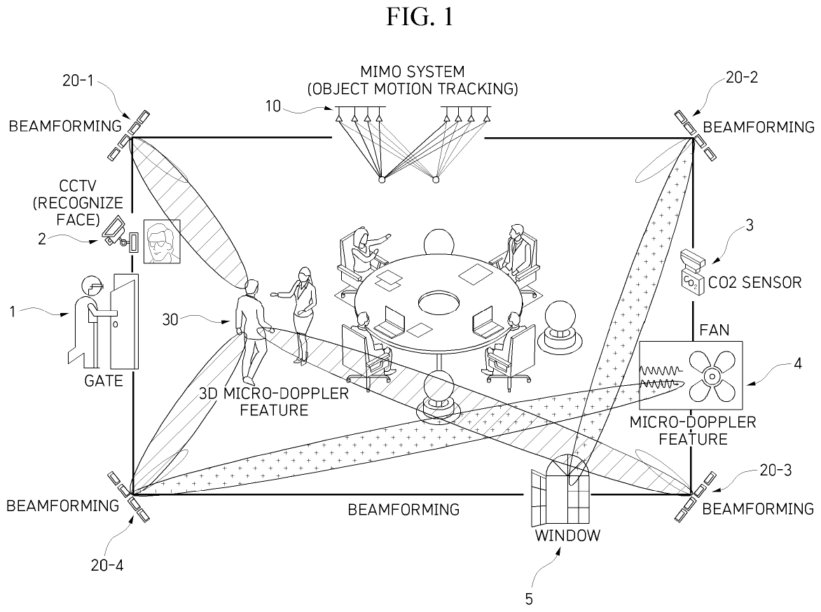

FIG. 1 illustrates an abnormality symptom detection service according to an embodiment of the present disclosure.

When an object (e.g., a person) 1 enters a specific space such as an office, identification information of the object 1 is obtained by recognizing the face of the object 1 through CCTV 2 that is installed at a gate.

A moving path of the object 1 who has entered the space is tracked by a MIMO system 10 that is installed at the ceiling. The MIMO system 10 transmits location information of the object 1 having a suspicious abnormality symptom to four beamforming antenna systems 20-1 to 20-4 that are installed on the sides of the space. In this case, the location information of the object is shared between the MIMO system 10 and the four beamforming antenna systems 20-1 to 20-4 through Ethernet communication.

Two or three beamforming antenna systems (e.g., 20-1, 20-3, and 20-4 in FIG. 1) line-of-sight environments of which have been secured, among the four beamforming antenna systems, form antenna radiation beams toward the object 1 having a suspicious abnormality symptom.

A degree of risk of the object having a suspicious abnormality symptom is determined and predicted by obtaining 3-D micro-Doppler information of the object 1 by using a plurality of beamforming antennas and constructing time-series data by using the obtained 3-D micro-Doppler information.

Whether devices 4 and 5, such as a window and a fan, properly operate is checked by measuring the air quality of the indoor space by using a sensor 3 (e.g., a CO₂ sensor is illustrated in FIG. 1, but the sensor 3 may be a temperature or humidity sensor).

Whether the window has been properly opened is determined based on a radar cross section (RCS) signal of a beamforming radar system. Whether the fan properly operates is determined based on a micro-Doppler signal of the beamforming radar system.

In order to maintain a clean indoor environment, a control signal for controlling an operation of the device (e.g., a fan or a window) is generated.

According to an embodiment of the present disclosure, an abnormality symptom of the object 1 can be detected more sophisticatedly by obtaining a micro-Doppler signal in the rear or on the side of the object 1, which cannot be obtained based on a 1-D micro-Doppler signal of the object 1, based on a 3-D micro-Doppler signal of the object 1.

According to an embodiment of the present disclosure, 3-D time-series data is constructed based on a 3-D micro-Doppler signal. A degree of risk of an object is determined and predicted by using a deep learning model based on time-series data.

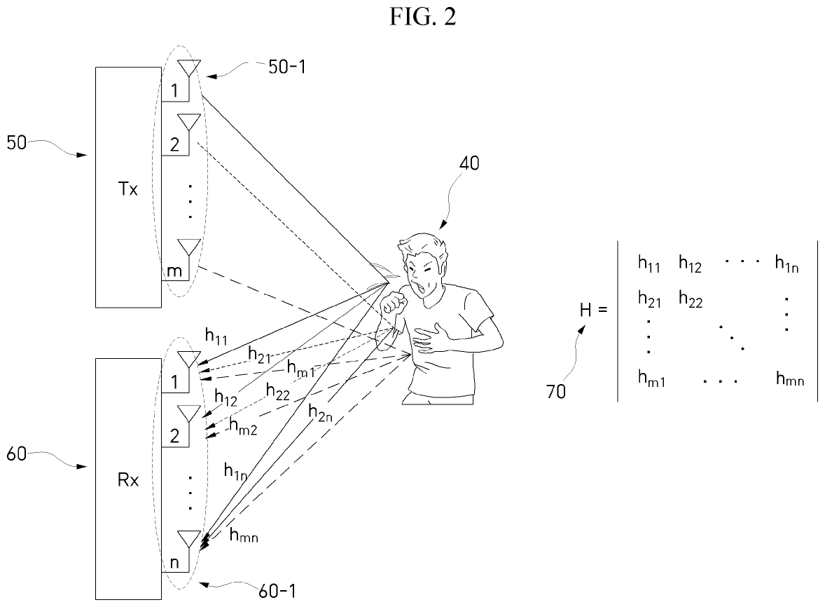

FIG. 2 illustrates the tracking of an object location and a channel transfer function matrix based on an MIMO system.

A transmitter 50 of the MIMO system includes m transmission antennas 50-1, and a receiver 60 thereof includes n reception antennas 60-1. A signal that is transmitted by a single antenna that constitutes each of the transmission antennas 50-1 is reflected by an object (e.g., a person) 40 and is received by a single antenna that constitutes each of the reception antennas 60-1.

In the MIMO system, signals that are transmitted by the plurality of transmission antennas 50-1 and that are received by the plurality of reception antennas 60-1 exhibit signal features based on a channel transfer function. In general, the channel transfer function may perform a high-speed operation by constructing a channel transfer function matrix 70. Moving paths of a plurality of objects (e.g., persons) may be tracked by using the channel transfer function matrix 70 of the MIMO system.

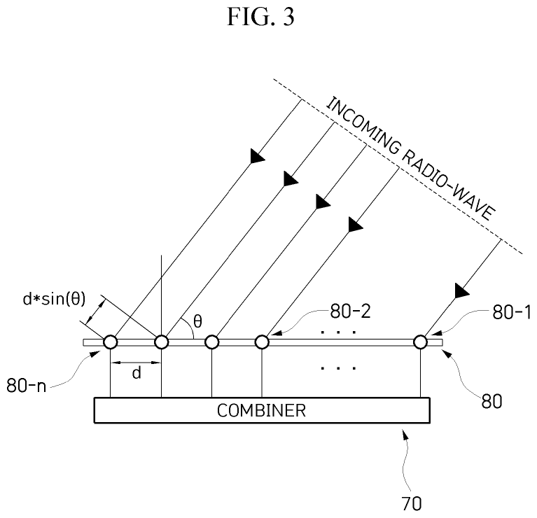

FIG. 3 illustrates the steering of beams of the beamforming antenna system.

An antenna 80 of the beamforming antenna system includes n antenna elements 80-1, 80-2 to 80-n in an array form, and changes a form of an antenna radiation beam by controlling a phase and amplitude of a current that is supplied to each of the antenna elements 80-1, 80-2 to 80-n. Accordingly, a main beam of the antenna 80 is formed.

As illustrated in FIG. 3, radio waves that are incident on the antenna 80 in which the antenna elements 80-1, 80-2 to 80-n are arranged at intervals of “d” in a direction of θ sequentially have delay times of d*sin(θ) on the basis of the antenna elements 80-1 that receives the radio wave first. Such delay times sequentially have phase delays of β*d*sin(θ) in the respective antenna elements. In this case, β is a propagation constant, and has a value of 2π/λ. Furthermore, λ indicates a wavelength of a center frequency.

Accordingly, a combiner 70 of the beamforming antenna system sequentially compensates for the phase delays of β*d*sin(θ) with respect to the antenna elements 80-1, 80-2 to 80-n, respectively. Accordingly, an antenna radiation beam capable of transmitting and receiving the strongest signals in the direction of θ is formed.

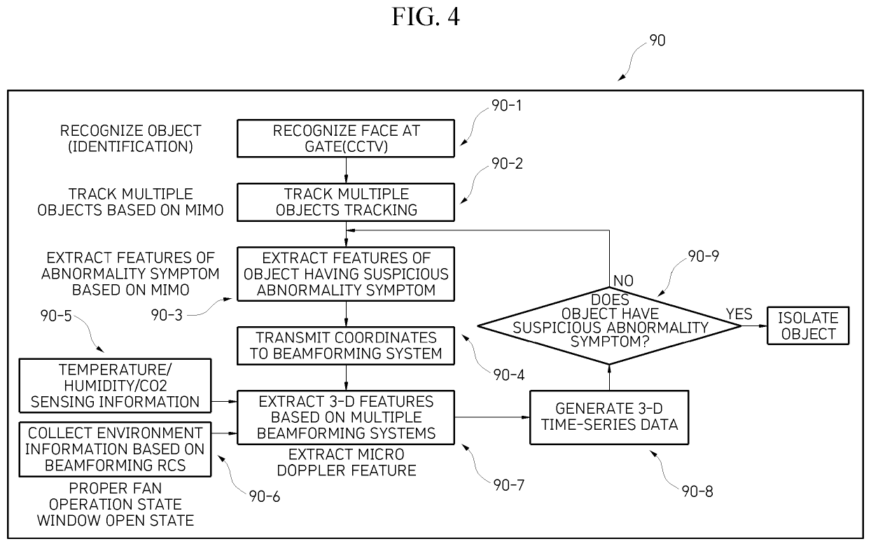

FIG. 4 illustrates a process 90 of detecting object abnormality based on a 3-D micro-Doppler signal according to an embodiment of the present disclosure.

In step 90-1, when an object (e.g., a person) enters a given space, identification information of the object (e.g., a person) is obtained by recognizing the face of the object (e.g., a person) through using CCTV.

In step 90-2, moving paths of multiple objects (e.g., persons) are tracked in real time based on the MIMO system.

In step 90-3, an object having a suspicious abnormality symptom, among the multiple objects, is detected based on a micro-Doppler signal. In step 90-4, coordinate information of the object having a suspicious abnormality symptom is transmitted to the beamforming antenna system.

In step 90-5, the beamforming antenna system collects environment information of the indoor space by using a sensor (e.g., a temperature, humidity, or CO₂ sensor). In step 90-6, the beamforming antenna system checks whether a device (e.g., a window or a fan) properly operates based on micro-Doppler information so that an indoor environment can be cleaned.

In step 90-7, the beamforming antenna system forms an antenna radiation beam toward the object having a suspicious abnormality symptom, and collects a 3-D micro-Doppler signal in order to detect a precise abnormality symptom of the object. The 3-D micro-Doppler signal is a signal on which periodic vibration (e.g., breathing, twitching, or chill) information, which may be detected in front of, on the side of, or in the rear of the object, can be interpreted in a frequency region.

In step 90-8, 3-D time-series data is constructed based on the micro-Doppler signals that are received from the plurality of beamforming antenna systems.

In step 90-9, the 3-D time-series data is used as input data for a deep learning model for predicting a degree of risk of the object and is used as data for predicting a degree of risk of the object. When it is confirmed that an object abnormality symptom is present, an object isolation process is performed.

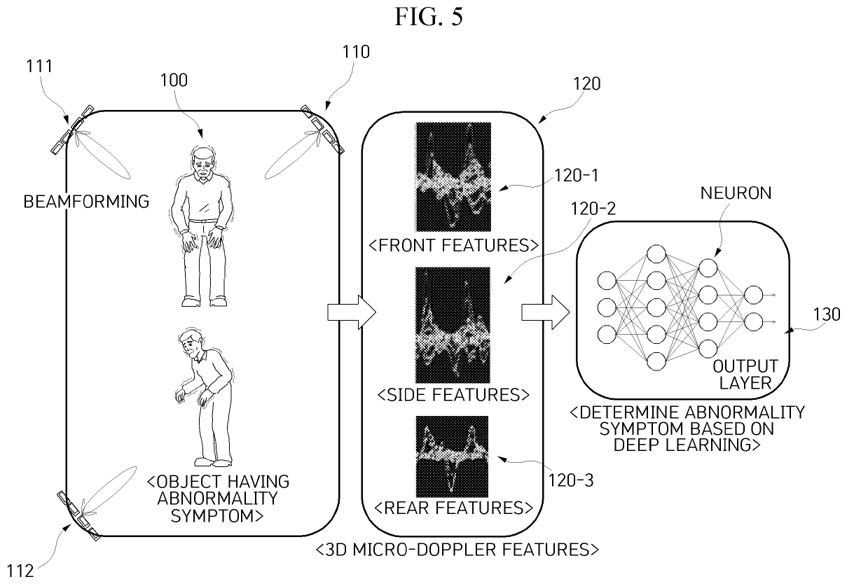

FIG. 5 illustrates the acquisition of a micro-Doppler signal of an object having a suspicious abnormality symptom and a deep learning model having input data including 3-D time-series data according to an embodiment of the present disclosure.

When an object 100 having a suspicious abnormality symptom enters a given space, a plurality of beamforming antenna systems 110, 111, and 112 forms antenna radiation beams toward the object 100 having a suspicious abnormality symptom.

The antenna radiation beam is reflected by the object 100 having a suspicious abnormality symptom and is then received. The object 100 having a suspicious abnormality symptom coughs or shivers due to the abnormality symptom. Accordingly, various micro-Doppler signals 120 are received from the head, the heart, arms, legs, etc. of the object 100 in a 3-D manner.

The micro-Doppler signals 120 constitute 3-D micro-Doppler information through a spectrum signal 120-1 at the front of the object, a spectrum signal 120-2 on the side of the object, and a spectrum signal 120-3 at the back of the object.

Each of the beamforming antenna systems 110, 111, and 112 that steer the front, side, and rear of the object extracts a meaningful micro-Doppler signal while scanning the whole body of the object 100. The beamforming antenna systems 110, 111, and 112 obtain change features of the abnormality symptom of the object 100 over time by collecting the micro-Doppler signals having time-series data features with respect to the object 100.

Since the micro-Doppler signal is generated in the frequency region, a change in the abnormality symptom of the object 100 is analyzed in a time axis through spectrogram analysis, such as a short time Fourier transform (STFT).

The abnormality symptom data obtained by the plurality of beamforming antennas 110, 111, and 112 may constitute 3-D time-series data, and is used as an input to a deep learning model 130 for determining and predicting an abnormality symptom.

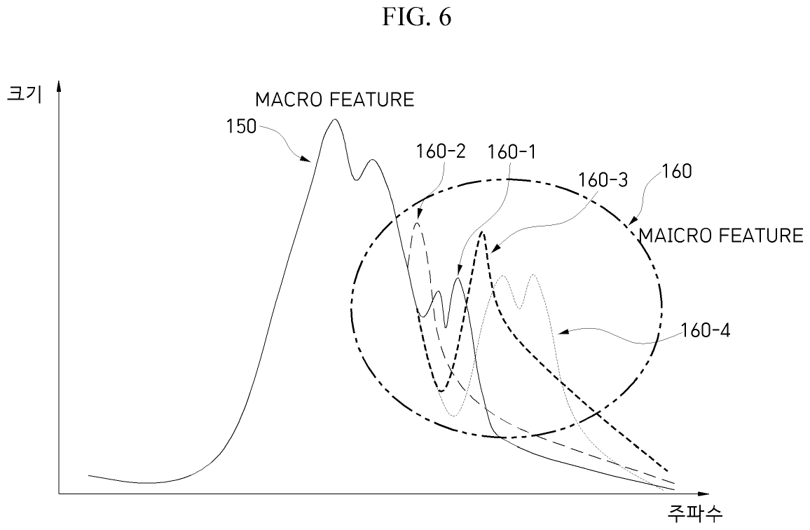

FIG. 6 illustrates an example of a time-series micro-Doppler signal of a radar.

A reception signal of the radar that is reflected by an object having a periodic motion and then received has two types of features. A macro-Doppler feature 150 is a feature that appears by a macroscopic and large motion of the object. A micro-Doppler feature 160 is a feature that appears by a fine motion of the object.

According to an embodiment of the present disclosure, the micro-Doppler feature 160 is used. An object having a suspicious abnormality symptom has the micro-Doppler feature 160 that is different due to a change over time.

For example, an object having a suspicious abnormality symptom may exhibit a micro-Doppler feature 160-1 in a time t=0, may exhibit a micro-Doppler feature 160-2 in a time t=2, may exhibit a micro-Doppler feature 160-3 in a time t=3, and may exhibit a micro-Doppler feature 160-4 in a time t=4.

As described above, a degree of risk of an abnormality symptom of an object is predicted by constructing a change in the features of a micro-Doppler frequency over time in the form of time-series data.

The time-series data is time-series data in which micro-Doppler frequency features are normalized by an average thereof and a standard deviation therebetween over time.

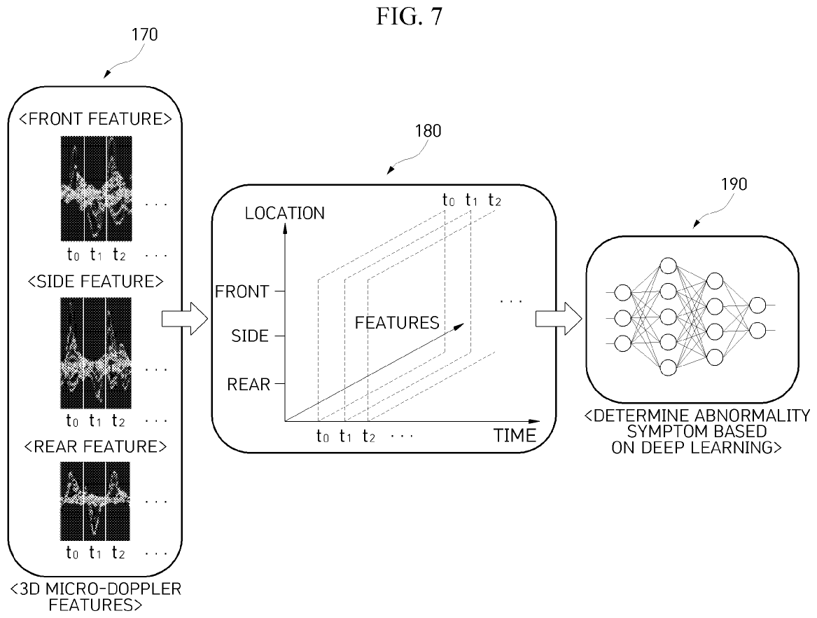

FIG. 7 illustrates an example in which 3-D micro-Doppler features are converted into time-series data.

As illustrated in FIG. 7, micro-Doppler signals that are reflected by an object having a suspicious abnormality symptom and then received may exhibit periodic patterns, but may exhibit different frequency patterns 170 over time depending on situations of the object.

Referring to reference numeral 180 in FIG. 7, three data (i.e., frequency features) collected in a time t0 are indicated on a time plane t0. Three data (i.e., frequency features) collected in a time t1 are indicated on a time plane t1. As described above, time-series frequency feature data over time are input to a deep learning model 190 in order to detect and predict an abnormality symptom of an object.

As described above, it is possible to increase the accuracy of a determination/prediction for an abnormality symptom of an object based on 3-D micro-Doppler signal time-series data.

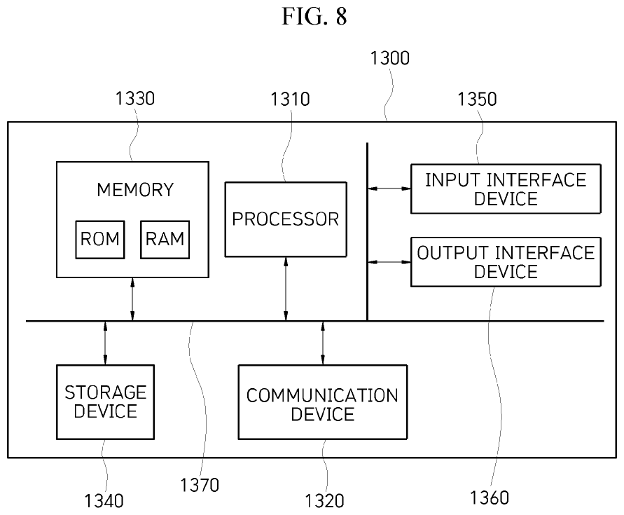

FIG. 8 is a block diagram illustrating a computer system for implementing a method according to an embodiment of the present disclosure.

Referring to FIG. 8, a computer system 1300 may include at least one of a processor 1310, memory 1330, an input interface device 1350, an output interface device 1360, and a storage device 1340 which communicate with each other through a bus 1370. The computer system 1300 may further include a communication device 1320 that is connected to a network. The processor 1310 may be a central processing unit (CPU) or may be a semiconductor device that executes an instruction stored in the memory 1330 or the storage device 1340. The memory 1330 and the storage device 1340 may include various types of volatile or nonvolatile storage media. For example, the memory may include read only memory (ROM) and random access memory (RAM). In an embodiment of this specification, the memory may be disposed inside or outside the processor, and may be connected to the processor through various known means. The memory includes various types of volatile or nonvolatile storage media, and may include ROM or RAM, for example.

Accordingly, an embodiment of the present disclosure may be implemented in the form of a method implemented in a computer, or may be implemented in the form of a non-transitory computer-readable medium in which a computer-executable instruction is stored. In one embodiment, when executed by a processor, the computer-executable instruction may perform a method according to at least one aspect of this specification.

The communication device 1320 may transmit or receive a wired signal or a wireless signal.

Furthermore, the method according to an embodiment of the present disclosure may be implemented in the form of a program instruction executable through various computer means, and may be recorded on a computer-readable medium.

The computer-readable medium may include a program instruction, a data file, and a data structure alone or in combination. The program instruction recorded on the computer-readable medium may be specially designed and constructed for an embodiment of the present disclosure, or may be known and available to those skilled in the computer software field. The computer-readable medium may include a hardware device configured to store and perform a program instruction. For example, the computer-readable medium may include magnetic media such as a hard disk, a floppy disk and a magnetic tape, optical media such as CD-ROM and a DVD, magneto-optical media such as a floptical disk, ROM, RAM, and a flash memory. The program instruction may include a high-level language code executable by a computer through an interpreter in addition to a machine-language code, such as that written by a compiler.

The embodiments of the present disclosure have been described above in detail, but the scope of rights of the present disclosure is not limited thereto and includes a variety of modifications and changes by those skilled in the art using the basic concept of the present disclosure which is defined in the appended claims.

What is claimed is:

1. A system for detecting an object abnormality symptom based on a radar micro-Doppler, the system comprising:
a multiple input multiple output (MIMO) system configured to determine whether an object has a suspicious abnormality symptom by collecting information of the object; and
a beamforming antenna system configured to form an antenna radiation beam toward the object based on a result of the determination for the object having the suspicious abnormality symptom and to obtain three-dimensional (3-D) micro-Doppler information.

2. The system of claim 1, wherein:
the MIMO system comprises a transmission antenna and a reception antenna, and
the MIMO system tracks a moving path of the object by using a channel transfer function in relation to a signal which is transmitted by the transmission antenna and received by the reception antenna.

3. The system of claim 1, wherein the beamforming antenna system determines a degree of risk of the object by forming the antenna radiation beam in a situation in which a line-of-sight has been secured, constructing time-series data by using the 3-D micro-Doppler information, and obtaining features of the abnormality symptom of the object which are changed over time.

4. The system of claim 3, wherein the beamforming antenna system determines the degree of risk of the object by a deep learning model using the time-series data as input data.

5. The system of claim 3, wherein the beamforming antenna system analyzes the changed features of the abnormality symptom of the object in a time axis by performing spectrogram analysis.

6. The system of claim 1, wherein the beamforming antenna system comprises a combiner configured to compensate for a phase delay based on an interval between antenna elements disposed in an array form, incident angle information of a radio wave, and a propagation constant.

7. The system of claim 1, further comprising a controller configured to receive a result of a determination for whether a device properly operates from the beamforming antenna system and to generate an operation control signal for the device based on the result of the determination.

8. A method of detecting an object abnormality symptom based on a radar micro-Doppler, the method comprising steps of:
(a) detecting an object that enters a space and tracking a moving path of the object; and
(b) forming an antenna radiation beam toward the object, collecting and analyzing a three-dimensional (3-D) micro-Doppler signal of the object, and confirming whether the object has an abnormality symptom.

9. The method of claim 8, wherein the step (a) comprises tracking the moving path by using a channel transfer function matrix of a multiple input multiple output (MIMO) system.

10. The method of claim 8, wherein the step (b) comprises confirming whether the object has the abnormality symptom in a way to form the antenna radiation beam by using a beamforming antenna system a line-of-sight of which has been secured and to construct time-series data based on the obtained 3-D micro-Doppler signal.

11. The method of claim 10, wherein the step (b) comprises forming the antenna radiation beam by compensating for phase delays based on an interval between antenna elements, an incident angle of a radio wave, and a wavelength of a center frequency.

12. The method of claim 8, further comprising a step (c) of checking whether a device within the space properly operates by using at least any one of sensing information, a radar cross-section, or the micro-Doppler signal and generating a control command signal for the device.
